(19) United States
(12) Patent Application Publication
(10) Pub. No.: US 2023/0355840 A1
(43) Pub. Date: Nov. 9, 2023

(54) MODIFIED PORCINE SCAFFOLDS AND METHODS OF PREPARATION

(71) Applicant: CONVATEC TRIAD LIFE SCIENCES, LLC., Memphis, TN (US)

(72) Inventors: Jon G. HARGIS, Memphis, TN (US); Kurt KLITZKE, Memphis, TN (US)

(21) Appl. No.: 17/311,468

(22) PCT Filed: May 26, 2021
(86) PCT No.: PCT/US2021/034198

§ 371 Date: Jun. 7, 2021

Related U.S. Application Data
(60) Provisional application No. 62/704,724, filed on May 26, 2020.

(51) Int. Cl. A61L 27/36; A61L 15/44; A61L 15/40

(52) U.S. Cl. CPC A61L 27/3633; A61L 15/44; A61L 15/40; A61L 2300/414; A61L 2300/252

(57) ABSTRACT

Porcine scaffolds are provided that include decellularized, porcine placental extracellular matrix that is processed so as to include one or more cytokines in an amount different from native porcine placental membrane. Methods of treating a wound are also provided.

### CROSS-REFERENCE TO RELATED APPLICATIONS

The present application is a national stage of PCT/US21/34198 filed May 26, 2021 which claims priority to U.S. Ser. No. 62/704,724 filed May 26, 2020, the contents of which are incorporated herein in their entirety.

### BACKGROUND

Extracellular matrices (ECMs) are the secreted molecules that create the microenvironment for cells and provide tissue with shape and strength (Brew, Dinakarpandian, & Nagase, 2000; Young, Holle, & Spatz, 2016). Biologic scaffolds created from extracellular matrix (ECM) are becoming increasingly common for the treatment of a variety of medical conditions (Hussey, Dziki, & Badylak, 2018). Commercially available scaffolds are created from a wide array of sources; multiple species and tissue types have been successfully processed into biologic scaffolds, including porcine small intestinal submucosa, bovine pericardium, porcine urinary bladder, and human dermis (Agmon & Christman, 2016).

In most commercially available ECMs, the structural component is primarily collagen, with the majority comprising type I collagen (Badylak, Freytes, & Gilbert, 2009). Additional fibril collagen species, types III, V and XI, and non-fibril collagen forms, types IV and VIII, will be present depending on the source material and tissue type (Theocharis, Skandalis, Gialeli, & Karamanos, 2016). In addition to the collagen content, ECMs can contain several adhesion molecules (Badylak et al., 2009) such as elastin, fibronectin, and laminin, which give each tissue type a unique ECM related to the tissue's function. The third major structural component of ECM (Badylak et al., 2009) are proteoglycans (PG), a base protein bonded with one or more glycosaminoglycans (GAG). Some common extracellular proteoglycans are aggrecan, versican, and decorin but, like other structural molecules, proteoglycans content varies based on source material and tissue type (Theocharis et al., 2016).

The functional component in ECM is a range of cytokines that contribute to its function via autocrine, juxtacrine and paracrine signaling (Wallner, 1998). The functional cytokines can be divided into categories based on action; there are growth factors, interleukins and tissue inhibitors of metalloproteinases (TIMP) (Engin, 2017; Hussey, 2018; Theocharis, 2016; Kim, 2011). Growth factors, which are signaling proteins that direct cellular activity, are present throughout the body and contribute to the proper function of tissue. Common growth factors include: vascular endothelial growth factor (VEGF), insulin-like growth factor (IGF), platelet derived growth factor (PDGF) and transforming growth factor-beta (TGFβ) (Kim, 2011; Taipale, 1997). Interleukins, a group of proteins primarily responsible for cell communication of the immune system, are critical for the control of both immune and inflammatory responses. There are more than 50 interleukins that have been identified (Brocker, 2010). TIMPs help regulate the function of tissue by inhibiting the activity of matrix metalloproteinases, a collection of enzymes that can degrade the various matrix proteins (Brew, 2000). All of the functional molecules work interactively to maintain normal and reparative activities in tissue (Schultz, 2011).

Studies have explored the role of multiple molecules in the wound healing process. Both Broughton and Li provide broad overviews of involvement of growth factors and ECM proteins and macromolecules in the wound healing process (Broughton, 2006; Li, 2007). A review specific to growth factors involved in wound healing was published by Dinh, et. al. that summarized work that demonstrated that TGFβ, PDGF, basic fibroblast growth factor (bFGF), and epidermal growth factor all play key roles in wound healing (Dinh, 2015). The multiple roles of VEGF as a cell mitogen, agent for chemotaxis and vascular permeability inducer were examined in a study by Bao, et. al (Bao, 2009). Studies have also examined the important role that collagen plays in wound repair (Brett, 2008; Madden, 1971). Hyaluronic acid was shown to play crucial roles in wound healing during inflammation and matrix synthesis (Aya, 2014; Chen, 1999). Both fibronectin (Grinnell, 1981; Sethi, 2002) and laminin (Malinda, 2008; Ishihara, 2018) have been shown to bind critical growth factors and enhance wound healing and cellular activity.

When creating a biologic scaffold for healing damaged tissue, many factors influence the effectiveness of the final scaffold; source material, tissue type, quality of the tissue source, site of the tissue, age of the donor, recovery process, decellularization, manufacturing process and sterilization.

Placental membrane, with its critical role of protection and nourishment during fetal development, provides many unique properties that make it an ideal source tissue for a biologic scaffold (Shaifur Ra, Islam, Asaduzzama, & Shahedur R, 2015). To that end, human placental membrane (e.g., amniotic membrane and/or chorion tissue) has been used for various types of reconstructive surgical procedures since the early 1900's. The membrane serves as a substrate material, more commonly referred to as a biological dressing or wound cover. Typically, human placental membranes are recovered after a cesarean section and are minimally processed, so that the manufacturing process does not alter the original relevant characteristics of the membrane relating to the membrane's utility for reconstruction, repair, or replacement.

A primary function of the placental membrane is to provide a physiologic barrier to prevent desiccation (Mamede et al., 2012) and an immunological barrier for the fetus. Placental membrane tissue exhibits anti-microbial properties due to the presence of β-3 defensins that act to prevent microbial colonization of the epithelial surface (Chopra & Thomas, 2013; Niknejad et al., 2008). Anti-inflammatory properties, based on the presence of interleukin-4 (IL-4), interleukin-10 (IL-10), TIMP-1, TIMP-2 and TIMP-4 (Hortensius & Harley, 2016; Mamede et al., 2012), of placental membranes are also beneficial to tissue healing. Placental membranes have shown clinical evidence of epithelialization (Dua, Gomes, King, & Maharajan, 2004; Subrahmanyam, 1995; Ward & Bennett, 1984) and the potential to heal without scarring (Leavitt et al., 2016).

The quality of the source material for biologic scaffolds can prove challenging as all naturally-occurring materials have some inherent variability (Cardinal, 2015). This challenge is especially prevalent in human source materials. Variation in genetic expression among individuals has been well documented (Genomes Project et al., 2010; International HapMap et al., 2010), and that genetic variation has been demonstrated in tissue in single individuals (O'Huallachain, Karczewski, Weissman, Urban, & Snyder, 2012). In addition to the genetic variability, the tissue can also be influenced by a multiplicity of environmental and behavioral risk factors. For example, recovered human amniotic tissue can be affected by maternal lifestyle (Day et al., 2015). Increased expression of cytochrome P450 enzymes, a family of enzymes responsible for metabolizing toxic compounds, is a marker of oxidative stress in the human tissue (Strolin-Benedetti, Brogin, Bani, Oesch, & Hengstler, 1999). Human placental tissues have been shown to have increased cytochrome P450 levels in smokers (Huuskonen et al., 2016), drug users (Paakki et al., 2000), mothers with BMI>30 (DuBois et al., 2012), diabetics (McRobie, Glover, & Tracy, 1998) and alcohol users (Collier, Tingle, Paxton, Mitchell, & Keelan, 2002). Maternal and gestational age have been shown to alter expression of cytochrome P450 (Collier et al., 2002) and growth factors (Lopez-Valladares et al., 2010) in human placental tissues.

Despite the wide variability associated with human placental products, in the last decade, rising awareness of the healing properties associated with such products has led to an increasing demand, thus propelling market growth. There remains a need in the art, however, for wound-healing treatments with high clinical efficiency that overcome the inconsistencies of the commercially available human-sourced products.

### SUMMARY OF THE DISCLOSURE

According to one aspect, a porcine scaffold is provided that includes decellularized, porcine placental extracellular matrix to form the porcine scaffold. The porcine scaffold includes at least one cytokine present in an amount different from native porcine placental membrane. According to one embodiment, the at least one cytokine is decorin present in an amount of at least about 70 pg/mg. According to one embodiment, the at least one cytokine is MIF present in an amount of less than about 50 pg/mg. According to one embodiment, the at least one cytokine is PDGF-BB present in an amount of at least about 75 pg/mg. According to one embodiment, the at least one cytokine is TIMP-2 present in an amount of less than about 175 pg/mg. According to one embodiment, the at least one cytokine is VEGF present in an amount of at least about 3 pg/mg. According to one embodiment, the at least one cytokine is PIGF-2 present in an amount of less than about 30 pg/mg. According to one embodiment, the at least one cytokine is TGF-β1 in an amount of at least about 82 pg/mg. According to one embodiment, the at least one cytokine is IGF-2 in an amount of at least about 7 pg/mg. According to one embodiment, the dehydrated porcine placental membrane is treated with a bioburden reduction step, a detergent rinse step, and a viral inactivation step.

According to one aspect, a porcine scaffold is provided that includes decellularized, porcine placental extracellular matrix to form the porcine scaffold. The porcine scaffold includes at least one cytokine for increasing vessel formation at a wound site. The porcine scaffold includes at least one cytokine present in an amount different from native porcine placental membrane. According to one embodiment, the at least one cytokine is decorin present in an amount of at least about 70 pg/mg. According to one embodiment, the at least one cytokine is VEGF present in an amount of at least about 3 pg/mg. According to one embodiment, the at least one cytokine is PIGF-2 present in an amount of less than about 30 pg/mg.

According to one aspect, a porcine scaffold is provided that includes decellularized, porcine placental extracellular matrix to form the porcine scaffold. The porcine scaffold includes at least one cytokine to regulate cell growth and division. The porcine scaffold includes at least one cytokine present in an amount different from native porcine placental membrane. According to one embodiment, the at least one cytokine is PDGF-BB present in an amount of at least about 75 pg/mg. According to one embodiment, the at least one cytokine is TGF-β1 in an amount of at least about 82 pg/mg. According to one embodiment, the at least one cytokine is IGF-2 in an amount of at least about 7 pg/mg.

According to one aspect, a porcine scaffold is provided that includes decellularized, porcine placental extracellular matrix to form the porcine scaffold. The porcine scaffold includes at least one cytokine that inhibits matrix metalloproteinase activity. The porcine scaffold includes at least one cytokine present in an amount different from native porcine placental membrane. According to one embodiment, the cytokine is TIMP-2 present in an amount of less than about 175 pg/mg.

According to one aspect, a porcine scaffold is provided that includes decellularized, porcine placental extracellular matrix to form the porcine scaffold. The porcine scaffold includes at least one cytokine for inflammatory modulation. The porcine scaffold includes at least one cytokine present in an amount different from native porcine placental membrane. According to one embodiment, the cytokine is MIF present in an amount of less than about 50 pg/mg. According to one aspect, a porcine scaffold is provided that includes decellularized, porcine placental extracellular matrix to form the porcine scaffold. The porcine scaffold includes at least one cytokine for stimulating collagen production. The porcine scaffold includes at least one cytokine present in an amount different from native porcine placental membrane. According to one embodiment, the cytokine is PIGF-2 present in an amount of less than about 30 pg/mg. A method of treating a defect is provided. The method includes the step of administering a porcine scaffold as provided herein to a defect. The defect may be a partial thickness wound, full thickness wound, pressure ulcer, venous ulcer, diabetic ulcer, chronic vascular ulcer, tunneled or undermined wound, surgical wound, wound dehiscence, abrasion, laceration, second degree burn, skin tear, draining wound, or any combination thereof.

A wound dressing is provided. The wound dressing includes a porcine scaffold as provided herein. According to one embodiment, the porcine scaffold includes a surface defining one or more fenestrations.

### DETAILED DESCRIPTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. As used in the specification, and in the appended claims, the words “optional” or “optionally” mean that the subsequently described event or circumstance can or cannot occur.

As used herein, the term “birth tissue” includes, but is not limited to, elements of mammalian birth tissue such as, for example, the placental membrane (amnion membrane and chorion membrane), Wharton's jelly, umbilical cord, umbilical artery, umbilical vein, and amniotic fluid.

As used herein, the term “commercialized ECM product” is a commercialized extracellular matrix product composed of a porcine small intestinal submucosa source material.

As used herein, the terms “fenestration” and “fenestrated” may be used interchangeably and refer to placental membrane-based constructs which have been further modified to include at least one or a plurality of prearranged through-holes (fenestrations) in the construct. Such holes allow exudate to traverse through the construct. Further, the number and size of the holes is predetermined so as to ensure that the fenestrations are appropriately spaced to provide sufficient opportunity for the exudate produced by a wound to pass through the construct, while also maintaining sufficient construct surface area to effectively treat the wound.

As used herein, the term “placental membrane” refers to the full, intact placental membrane including the amnion and chorion layers that are obtained from a mammal such as, for example, a pig or human.

As used herein, the term “membrane” refers to at least one placental membrane, at least one amnion membrane, at least one chorion membrane or any combination thereof. The membranes as referred to herein may be obtained from a mammal such as, for example, a pig or human.

As used herein, the terms “pig” and “porcine” may be used interchangeably.

As used to herein, the terms “porcine scaffold” and “powder-based construct” refer to a construct that is applied onto or around an injured area of a mammalian body.

As used herein, the terms “defect” and “wound” may be used interchangeably and refer to an area in need of treatment such as an injured area of the mammalian body.

As used herein, the term “decellularization” refers to the process by which all or substantially all of the intact cells and nuclei are removed from the placental membrane, leaving a placental extracellular matrix derived from original placental membrane.

As used herein, the term “placental extracellular matrix” refers to the decellularized placental membrane whereby all or substantially all of the intact cells and nuclei are removed from the placental membrane, leaving a three-dimensional network consisting of extracellular macromolecules, such as collagen, elastin, glycosaminoglycans, laminin, and fibronectin, that provide both structural and biochemical support when used in the patient's body.

As used herein, the term “scaffold” refers to a decellularized extracellular matrix structure that allows the patient's cells to infiltrate and aid in the healing cascade and regeneration of damaged tissue. The scaffolds provided herein include extracellular matrices derived from birth tissue such as porcine placental membrane or other mammalian placental membrane.

As used herein, the term “native” refers to the condition of a tissue after procuring from a mammal, such as a pig, but prior to being subject to the preparation steps provided herein.

The present disclosure provides an extracellular matrix scaffold that is prepared from mammalian birth tissue. The present disclosure particularly provides a porcine scaffold that is prepared from pig birth tissue. The methods and uses provided herein may be applied, however, to any mammalian-sourced birth tissue to form a scaffold suitable for regenerative purposes. Suitable mammals include, but are not limited to, human, bovine, equine, goat, or sheep.

Porcine placenta provides a unique source material for an extracellular matrix scaffold, while overcoming inherent challenges associated with human placenta. Social factors which seriously impact the availability and quality of human sourced tissue (e.g., obesity, tobacco, alcohol and drug consumption) are absent in porcine placenta. In contrast, purpose-bred sows have an entirely regimented life where the sows' age, diet, exercise regimen/activity level, and health care are in the complete control of the breeder, thereby reducing the variability of the porcine placental starting material. For example, unlike the widely-adopted age criteria for human placental membranes which encompasses all women of childbearing years regardless of age, sows are bred from the age of one until about the age of six, which corresponds to a human age of range of 16 to 35. Sows birth the piglets at about 114 days of gestation. The combination of low maternal and gestational age lessen the chance of stress markers (e.g., cytochrome P450 enzymes) to be present in porcine tissue.

The porcine scaffolds and methods provided herein seek to address the unmet need in the current human placental membrane market by providing an alternate source material that overcomes inherent challenges associated with human placenta noted herein. Aside from the fact that the porcine-sourced material does not have the same age, health and lifestyle issues that may impact human products, the present porcine scaffolds allow host cells to infiltrate the scaffold and affected area (e.g., defect), deposit collagen, and easily and quickly remodel the defect.

The porcine scaffolds as provided herein may aid in the healing cascade or healing process of a mammalian defect such as a wound or ulcer. The porcine scaffolds may be fully resorbed by the mammal's body during the healing process. Methods for aseptically processing placental membrane to prepare porcine scaffolds are provided. According to one embodiment, the porcine placental extracellular matrices are prepared from placental membranes that remain intact in that the placental membranes retain the amnion and chorion membrane layers and any intermediate layers. According to one embodiment, the placental membrane is processed in a manner such that all native layers are retained except for the Wharton's Jelly.

The porcine scaffolds as provided herein may be formulated as a membrane-based construct. According to one embodiment, the porcine scaffold includes one or more layers of porcine placental membrane including the full, intact placental membrane or one or more layers of isolated amnion or chorion. According to one embodiment, the porcine placental membrane includes dehydrated, decellularized porcine placental extracellular matrix as provided herein. According to another embodiment, the porcine scaffold as provided herein may also be formulated as a powder, gel, liquid or spray.

According to one embodiment, when formulated as a membrane-based scaffold, the placental membrane chosen to be processed to form the scaffold may be treated to provide for the delivery of a variety of antibiotics, anti-inflammatory agents, growth factors and/or other specialized proteins or small molecules. In addition, the resulting membrane-based scaffold may be combined with or covered by a substrate (sterile gauze, sterile polymer material or other tissue or biomaterial) to increase the strength of the porcine scaffold for sutures or to increase the longevity of an implant.

A scaffold as described herein may be produced by processing mammalian birth tissue according to any or all of the steps provided herein as applied to birth tissue. According to a particular embodiment, a porcine scaffold as described herein may be produced by processing pig birth tissue according to the steps provided herein.

A porcine scaffold is provided. According to one embodiment, the porcine scaffold includes decellularized, porcine placental extracellular matrix that includes one or more of collagen I, collagen III, collagen IV, elastin, laminin, fibronectin, hyaluronic acid and sulfated glycosaminoglycans. According to one embodiment, each of the one or more of collagen I, collagen III, collagen IV, elastin, laminin, fibronectin, hyaluronic acid and sulfated glycosaminoglycans is present in in an amount that is different from native porcine placental membrane that is not processed according to one or more of the processing steps provided herein. According to one embodiment, the porcine scaffold includes four major extracellular matrix components: collagen, elastin, hyaluronic acid and sulfated glycosaminoglycans (sGAGs). According to one embodiment, the porcine scaffold includes at least one or more cytokines in an amount different from that found in unmodified, unprocessed or native porcine birth tissue. Such cytokines include, but are not limited to, Eotaxin-1, EPO, FGF-21, Galectin-9, IFNb, IGF-2, IL-21, IL-28B, PIGF-2, SCF, ANG-1, IL-17F, MIF, OPG, PDGF-BB, RANTES, TGFa, TIMP-1, TIMP-2, VEGF, Decorin, GASP-1, IGFBP-5, IL-15, IL-22, Insulin, IP-10, MCP-1, NCAM-1, TWEAK R, CCL3L1, IFNa, IL-1a, IL-1ra, IL-13, IL-17a, IL-18, MIG, MIP-1b, PECAM-1, IL-1b, IL-4, IL-6, IL-8, IL-10, IL-12p40p70, GM-CSF, IFNg, TGF-β1, and TNFa.

A porcine scaffold is provided that includes decellularized, porcine placental extracellular matrix to form the porcine scaffold. The porcine scaffold includes at least one cytokine present in an amount different from native porcine placental membrane.

According to one embodiment, the at least one cytokine is decorin present in an amount of at least about 70 pg/mg. According to one embodiment, the at least one cytokine is decorin present in an amount of at least about 75 pg/mg. According to one embodiment, the at least one cytokine is decorin present in an amount of at least about 80 pg/mg. According to one embodiment, the at least one cytokine is decorin present in an amount of at least about 85 pg/mg. According to one embodiment, the at least one cytokine is decorin present in an amount of at least about 90 pg/mg. According to one embodiment, the at least one cytokine is decorin present in an amount of at least about 95 pg/mg. According to one embodiment, the at least one cytokine is decorin present in an amount of at least about 100 pg/mg.

According to one embodiment, the at least one cytokine is MIF present in an amount of less than about 50 pg/mg. According to one embodiment, the at least one cytokine is MIF present in an amount of less than about 40 pg/mg. According to one embodiment, the at least one cytokine is MIF present in an amount of less than about 30 pg/mg. According to one embodiment, the at least one cytokine is MIF present in an amount of less than about 20 pg/mg. According to one embodiment, the at least one cytokine is MIF present in an amount of less than about 10 pg/mg. According to one embodiment, the at least one cytokine is MIF present in an amount of less than about 5 pg/mg.

According to one embodiment, the at least one cytokine is PDGF-BB present in an amount of at least about 75 pg/mg. According to one embodiment, the at least one cytokine is PDGF-BB present in an amount of at least about 80 pg/mg. According to one embodiment, the at least one cytokine is PDGF-BB present in an amount of at least about 85 pg/mg. According to one embodiment, the at least one cytokine is PDGF-BB present in an amount of at least about 90 pg/mg. According to one embodiment, the at least one cytokine is PDGF-BB present in an amount of at least about 95 pg/mg.

According to one embodiment, the at least one cytokine is TIMP-2 present in an amount of less than about 175 pg/mg. According to one embodiment, the at least one cytokine is TIMP-2 present in an amount of less than about 150 pg/mg. According to one embodiment, the at least one cytokine is TIMP-2 present in an amount of less than about 125 pg/mg. According to one embodiment, the at least one cytokine is TIMP-2 present in an amount of less than about 100 pg/mg. According to one embodiment, the at least one cytokine is TIMP-2 present in an amount of less than about 75 pg/mg. According to one embodiment, the at least one cytokine is TIMP-2 present in an amount of less than about 50 pg/mg. According to one embodiment, the at least one cytokine is TIMP-2 present in an amount of less than about 25 pg/mg. According to one embodiment, the at least one cytokine is TIMP-2 present in an amount of less than about 12 pg/mg. According to one embodiment, the at least one cytokine is TIMP-2 present in an amount of less than about 6 pg/mg. According to one embodiment, the at least one cytokine is TIMP-2 present in an amount of less than about 3 pg/mg.

According to one embodiment, the at least one cytokine is VEGF present in an amount of at least about 3 pg/mg. According to one embodiment, the at least one cytokine is VEGF present in an amount of at least about 10 pg/mg. According to one embodiment, the at least one cytokine is VEGF present in an amount of at least about 20 pg/mg. According to one embodiment, the at least one cytokine is VEGF present in an amount of at least about 40 pg/mg. According to one embodiment, the at least one cytokine is VEGF present in an amount of at least about 80 pg/mg. According to one embodiment, the at least one cytokine is VEGF present in an amount of at least about 150 pg/mg.

According to one embodiment, the at least one cytokine is PIGF-2 present in an amount of less than about 30 pg/mg. According to one embodiment, the at least one cytokine is PIGF-2 present in an amount of less than about 25 pg/mg. According to one embodiment, the at least one cytokine is PIGF-2 present in an amount of less than about 20 pg/mg. According to one embodiment, the at least one cytokine is PIGF-2 present in an amount of less than about 15 pg/mg. According to one embodiment, the at least one cytokine is PIGF-2 present in an amount of less than about 7 pg/mg.

According to one embodiment, the at least one cytokine is TGF-β1 in an amount of at least about 82 pg/mg. According to one embodiment, the at least one cytokine is TGF-β1 in an amount of at least about 100 pg/mg. According to one embodiment, the at least one cytokine is TGF-β1 in an amount of at least about 200 pg/mg. According to one embodiment, the at least one cytokine is TGF-β1 in an amount of at least about 300 pg/mg. According to one embodiment, the at least one cytokine is TGF-β1 in an amount of at least about 400 pg/mg. According to one embodiment, the at least one cytokine is TGF-β1 in an amount of at least about 500 pg/mg. According to one embodiment, the at least one cytokine is TGF-β1 in an amount of at least about 600 pg/mg. According to one embodiment, the at least one cytokine is TGF-β1 in an amount of at least about 700 pg/mg.

According to one embodiment, the at least one cytokine is IGF-2 in an amount of at least about 7 pg/mg. According to one embodiment, the at least one cytokine is IGF-2 in an amount of at least about 10 pg/mg. According to one embodiment, the at least one cytokine is IGF-2 in an amount of at least about 100 pg/mg. According to one embodiment, the at least one cytokine is IGF-2 in an amount of at least about 200 pg/mg. According to one embodiment, the at least one cytokine is IGF-2 in an amount of at least about 300 pg/mg. According to one embodiment, the at least one cytokine is IGF-2 in an amount of at least about 400 pg/mg.

According to one embodiment, the dehydrated porcine placental membrane is treated with a bioburden reduction step, a detergent rinse step, and a viral inactivation step.

A porcine scaffold is provided that includes decellularized, porcine placental extracellular matrix to form the porcine scaffold. The porcine scaffold includes at least one cytokine for increasing vessel formation at a wound site. The porcine scaffold includes at least one cytokine present in an amount different from native porcine placental membrane. According to one embodiment, the at least one cytokine is decorin present in an amount of at least about 70 pg/mg, 75 pg/mg, 80 pg/mg, 85 pg/mg, 90 pg/mg, 95 pg/mg or 100 pg/mg. According to one embodiment, the at least one cytokine is VEGF present in an amount of at least about 3 pg/mg, 10 pg/mg, 20 pg/mg, 40 pg/mg, 80 pg/mg, or 150 pg/mg. According to one embodiment, the at least one cytokine is PIGF-2 present in an amount of less than about 30 pg/mg, 25 pg/mg, 20 pg/mg, 15 pg/mg, or 7 pg/mg. According to one embodiment, the at least one cytokine is a combination of one or more of decorin, VEGF, and PIGF-2 in the aforementioned amounts.

A porcine scaffold is provided that includes decellularized, porcine placental extracellular matrix to form the porcine scaffold. The porcine scaffold includes at least one cytokine to regulate cell growth and division. The porcine scaffold includes at least one cytokine present in an amount different from native porcine placental membrane. According to one embodiment, the at least one cytokine is PDGF-BB present in an amount of at least about 75 pg/mg, 80 pg/mg, 85 pg/mg, 90 pg/mg or 95 pg/mg. According to one embodiment, the at least one cytokine is TGF-β1 in an amount of at least about 82 pg/mg, 100 pg/mg, 200 pg/mg, 300 pg/mg, 400 pg/mg, 500 pg/mg, 600 pg/mg or 700 pg/mg. According to one embodiment, the at least one cytokine is IGF-2 in an amount of at least about 7 pg/mg, 10 pg/mg, 100 pg/mg, 200 pg/mg, 300 pg/mg or 400 pg/mg. According to one embodiment, the at least one cytokine is a combination of one or more of PDGF-BB, TGF-β1, or IGF-2 in the aforementioned amounts.

A porcine scaffold is provided that includes decellularized, porcine placental extracellular matrix to form the porcine scaffold. The porcine scaffold includes at least one cytokine that inhibits matrix metalloproteinase activity. The porcine scaffold includes at least one cytokine present in an amount different from native porcine placental membrane. According to one embodiment, the cytokine is TIMP-2 present in an amount of less than about 175 pg/mg, 100 pg/mg, 50 pg/mg, 25 pg/mg, 12 pg/mg, 6 pg/mg or 3 pg/mg.

A porcine scaffold is provided that includes decellularized, porcine placental extracellular matrix to form the porcine scaffold. The porcine scaffold includes at least one cytokine for inflammatory modulation. The porcine scaffold includes at least one cytokine present in an amount different from native porcine placental membrane. According to one embodiment, the cytokine is MIF present in an amount of less than about 50 pg/mg, 40 pg/mg, 30 pg/mg, 20 pg/mg, 10 pg/mg, or 5 pg/mg

A porcine scaffold is provided that includes decellularized, porcine placental extracellular matrix to form the porcine scaffold. The porcine scaffold includes at least one cytokine for stimulating collagen production. The porcine scaffold includes at least one cytokine present in an amount different from native porcine placental membrane. According to one embodiment, the cytokine is PIGF-2 present in an amount of less than about 30 pg/mg, 25 pg/mg, 20 pg/mg, 15 pg/mg, or 7 pg/mg.

Any of the porcine scaffolds provided herein may include a surface defining one or more fenestrations.

A wound dressing is also provided. The wound dressing includes a porcine scaffold as provided herein. According to one embodiment, the wound dressing may include a surface defining one or more fenestrations. According to one embodiment, the wound dressing may be combined with or covered by a substrate or non-adherent secondary dressing (sterile gauze, sterile polymer material or other tissue or biomaterial) to increase the strength of the porcine scaffold for sutures or to increase the longevity of an implant.

A method of preparing a porcine scaffold is provided. According to one embodiment, the step of processing the porcine placental membrane to form a porcine scaffold includes treating the porcine placental membrane with a detergent solution, the detergent solution including at least one protease enzyme. According to one embodiment, the detergent solution further includes at least one anionic detergent. According to one embodiment, the step of processing the porcine placental membrane to form a porcine scaffold includes treating the porcine placental membrane with a viral inactivation solution, the viral inactivation solution including at least one alkali solution. According to one embodiment, the alkali solution includes sodium hydroxide in an amount of about 1 mL to about 50 mL of about 0.1M to about 3.0M sodium hydroxide per gram of porcine placental membrane.

According to one embodiment, a method of preparing a porcine scaffold is provided. The method includes the step of collecting the birth tissue, including the umbilical cord, placental membrane (amnion and chorion membrane), and amniotic fluid from a female pig. According to one embodiment, the method includes the step of collecting the birth tissue, including the umbilical cord, placental membrane (amnion and chorion membrane), and amniotic fluid from a female pig. According to one embodiment, the female pig is not genetically modified to halt or reduce expression of the functional alpha-1,3 galactosyltransferase gene. According to one embodiment, the placental membrane includes the umbilical cord attached. Potential birth tissue donors are screened and tested to exclude any donors that may present a health risk. According to one embodiment, birth tissue is recovered from a full-term delivery of one or more offspring such as an infant or piglet(s). According to one embodiment, the method optionally includes the step of rinsing the birth tissue, including the umbilical cord and placental membrane (amnion and chorion membrane) by methods known to those skilled in the art. According to one embodiment, the method further includes the step of placing the birth tissue, including the umbilical cord and placental membrane (amnion and chorion membrane), in a transport container.

According to one embodiment, the method optionally includes the step of freezing the umbilical cord and placental membrane by methods known to those skilled in the art. According to one embodiment, the umbilical cord and placental membrane may be kept frozen until further processing is needed. According to one embodiment, the method further includes the step of removing the frozen, bagged umbilical cord and placental membrane from the freezer and thawing in a refrigerator. According to one embodiment, the method further includes the step of thawing the umbilical cord and placental membrane at ambient temperature. According to one embodiment, the method optionally includes the step of placing any retained, frozen amniotic fluid in a container.

According to one embodiment, the method includes rinsing the umbilical cord and placental membrane with water. According to one embodiment, the method includes draining the umbilical cord and placental membrane. According to one embodiment, the method includes separating the placental membrane from the umbilical cord.

According to one embodiment, the method includes the step of dividing the placental membrane into pieces. According to one embodiment, a rotary cutter or other suitable cutter is used to cut the pieces. When formulated as a membrane-based construct, the birth tissue may be cut to various sizes, thickness, and shapes. The placental membrane pieces are preferably of sufficient size and shape to be applied onto or around a wound that is on or in a mammalian patient's body. The placental membrane thickness may vary depending on application, the type of membrane and the number of membrane layers.

According to one embodiment, the method includes the step of removing Wharton's jelly and excess fluids from the placental membrane to produce cleaned placental membrane. According to one embodiment, the method includes the step of treating the placental membrane with a bioburden reduction solution. According to a preferred embodiment, the bioburden reduction solution is sodium chloride. According to one embodiment, the method includes the step of adding from about 1 mL to about 100 mL of 0.1M to about 5M sodium chloride solution per gram of placental membrane. According to one embodiment, the method includes the step of immersing the placental membrane in the sodium chloride solution from about fifteen minutes to about eight hours. According to one embodiment, the method includes the step of shaking the placental membrane in the sodium chloride solution from about fifteen minutes to about eight hours at about 20 RPM to about 100 RPM.

According to one embodiment, the method includes the step of decanting the sodium chloride. According to one embodiment, the method includes the step of rinsing the placental membrane with water. According to one embodiment, the placental membrane is rinsed one time with water. According to one embodiment, the rinsing step is carried out multiple times with water. According to one embodiment, the placental membrane is rinsed from about two times to about five times with water.

According to one embodiment, the method includes the step of placing the placental membrane in from about 1 mL to about 100 mL of a detergent solution. According to one embodiment, the detergent is present at a concentration of about 0.1% to about 10% w/v. According to one embodiment, the detergent solution includes at least one ionic detergent. According to a particular embodiment, the detergent solution includes at least one anionic detergent. According to a particular embodiment, the detergent solution includes at least one anionic detergent and at least one protease enzyme. According to one embodiment, the detergent solution contains phosphates, with a phosphorus content of about 7.5%. According to one embodiment, the detergent solution includes phosphates, carbonates, sodium linear alkylaryl sulfonate and at least one protease enzyme. According to one embodiment, the method includes the step of immersing the placental membrane in the detergent solution for from about fifteen minutes to about eight hours. According to one embodiment, the method includes the step of shaking the placental membrane in the detergent solution for from about fifteen minutes to about eight hours at about 20 RPM to about 100 RPM.

According to one embodiment, the method includes the step of decanting the detergent solution. According to one embodiment, the method includes the step of rinsing the placental membrane with water. According to one embodiment, the placental membrane is rinsed one time with water. According to one embodiment, the placental membrane is rinsed multiple times with water. According to one embodiment, the placental membrane is rinsed from about two times to about five times with water.

According to one embodiment, the method includes the step of treating the placental membrane with a viral inactivation solution, such as, for example, sodium hydroxide, hydrogen peroxide, ethanol or supercritical carbon dioxide. According to a preferred embodiment, the viral inactivation solution is sodium hydroxide. According to one embodiment, the method includes the step of adding or introducing from about 1 mL to about 50 mL of about 0.1M to about 3.0M sodium hydroxide per gram of placental membrane. According to one embodiment, the method includes the step of immersing the placental membrane in the sodium hydroxide for about 1 minute to about 120 minutes. According to one embodiment, the method includes the step of shaking the placental membrane in the sodium hydroxide for about 1 minute to about 120 minutes at about 20 RPM to about 100 RPM. The sodium hydroxide may then be decanted. According to one embodiment, the steps of adding sodium hydroxide, shaking and decanting may be repeated as many times as necessary to inactivate any viruses present in the placental membrane to produce a placental membrane that is substantially void of viruses. According to one embodiment, the steps of adding sodium hydroxide, shaking and decanting may be repeated once. According to one embodiment, the steps of adding sodium hydroxide, shaking and decanting may be repeated up to five times. According to a preferred embodiment, the method includes the step of adding or introducing from about 5 mL to about 15 mL of 0.25M sodium hydroxide per gram of placental membrane. According to a preferred embodiment, the method includes the step of adding or introducing about 10 mL of 0.25M sodium hydroxide for about 20 minutes, shaking, decanting and repeating the process one time. According to this preferred embodiment, the resulting porcine scaffold is substantially void of viruses, but much of the extracellular matrix composition is preserved, including a substantial percentage of the glycosaminoglycans. According to one embodiment, the method includes the step of rinsing the placental membrane with water.

According to one embodiment, the method includes the step of adding or introducing from about 1 mL to about 50 mL of buffer solution per gram of placental membrane. According to one embodiment, the method includes the step of immersing the placental membrane in the buffer solution. According to one embodiment, the method includes the step of shaking the placental membrane in the buffer solution for about 1 minute to about 120 minutes at about 20 RPM to about 100 RPM. The buffer solution may then be decanted. According to a preferred embodiment, the buffer solution is phosphate buffer solution. According to one embodiment, the method includes the step of measuring the pH of the placental membrane after buffer solution treatment. According to one embodiment, the steps of adding buffer solution, shaking and decanting may be repeated until the pH of the placental membrane is between about 6.8 and about 7.2.

According to one embodiment, the method includes the step of rinsing the placental membrane with water. According to one embodiment, the placental membrane is washed one time with water. According to one embodiment, the rinsing step is carried out multiple times with water. According to one embodiment, the placental membrane is rinsed from about two times to about five times with water.

When preparing a membrane-based construct, the placental membrane may be wet or dehydrated. According to one embodiment, the placental membrane may be dehydrated by any method known in the art, including, but not limited to, chemical dehydration (e.g., organic solvents), lyophilization, desiccation, oven dehydration and air drying. According to a preferred embodiment, the method includes the step of adding or introducing an alcohol to the placental membrane to cover the entire surface of the placental membrane (i.e., submerge the placental membrane). According to one embodiment, the method includes the step of adding or introducing from about 1 mL to about 100 mL of alcohol per gram of placental membrane. According to one embodiment, the placental membrane is fully submerged in the alcohol for from about ten minutes to about 24 hours. The alcohol may be any alcohol-safe and appropriate for contact with placental membrane. According to a particular embodiment, the alcohol is ethanol. According to one embodiment, the method includes the step of decanting or draining the alcohol from the placental membrane.

According to one embodiment, the method includes the step of spreading the placental membrane onto a drying table (e.g., a Delrin drying table). According to one embodiment, the placental membrane may be blotted with a micro fiber wipe or similar. The placental membrane may be spread in a manner so as to fully dehydrate the placental membrane while ensuring no wrinkles or bubbles are present.

When preparing a membrane-based construct, the method includes the step of cutting the placental membrane to a predetermined or desired size. According to one embodiment, the placental membrane is cut to size with a rotary cutter or other suitable instrument. According to one embodiment, the cuts are made with a scalpel blade.

According to another embodiment, the method includes the step of forming one or more (e.g., a plurality) of fenestrations in the placental membrane. Thus, the resulting scaffold includes a surface defining one or more fenestrations (e.g., through holes). According to one embodiment, the placental membrane may be fenestrated with a scalpel blade or other apparatus, with the one or more fenestrations appropriately spaced to provide sufficient opportunity for the exudate produced by a wound to pass through the placental membrane, while also maintaining sufficient placental membrane surface area to effectively treat a defect such as a wound or ulcer.

The processing methods provided herein result in a mammalian scaffold that includes a decellularized, placental extracellular matrix such as a decellularized, porcine placental extracellular matrix that is derived from placental membrane.

According to one embodiment, the method includes the step of placing the cut scaffold in one or more packaging materials.

According to one embodiment, the method includes the step of terminally sterilizing the packaged scaffold. According to one embodiment, the method of terminal sterilization may be e-beam irradiation, gamma irradiation, peracetic acid treatment, vaporized peracetic acid (VPA) treatment, any combination thereof, or any other terminal sterilization method known in the art. According to another embodiment, the scaffold is formulated as a powder-based construct. When preparing the powder-based construct, the scaffold may be wet or dehydrated. According to one embodiment, the scaffold may be dehydrated by any method known in the art, including, but not limited to, chemical dehydration (e.g., organic solvents), lyophilization, desiccation, oven dehydration and air drying. In one embodiment, the method includes the step of cutting the scaffold into a plurality of strips. According to one embodiment, the strips of scaffold may then be placed into a mill and ground into a powder to form a powder-based construct. According to one embodiment, scaffold may consist of the whole placental membrane or a portion thereof, which may be placed into a mill and ground into a powder to form a powder-based construct. According to one embodiment, the powder-based construct may then be placed into appropriate containers or vials at a desired concentration. According to one embodiment, the method of preparing a powder-based construct includes one or more steps of lyophilizing the milled/ground powder-based construct within the vials to remove residual moisture. Then, the vials containing the powder-based construct are terminally sterilized. According to one embodiment, the method of terminal sterilization may be e-beam irradiation, gamma irradiation, peracetic acid treatment, vaporized peracetic acid (VPA) treatment, any combination thereof, or any other terminal sterilization method known in the art.

A method of treating a defect is also provided. According to one embodiment, the method includes the step of providing a porcine scaffold as provided herein. The porcine scaffold is then administered (e.g., placed on or around) a defect. The defect may be a soft tissue defect including a wound such as, for example, a burn, cut, or abrasion. According to one embodiment, the defect is selected from a partial thickness wound, full thickness wound, pressure ulcer, venous ulcer, diabetic ulcer, chronic vascular ulcer, tunneled or undermined wound, surgical wound, wound dehiscence, abrasion, laceration, second degree burn, skin tear, and draining wound. The defect may also be any ulcer. According to one embodiment, the wound may be a surgical site anywhere on or in a mammalian body. The porcine scaffold may be placed over a surgical site or held in place by a patient's musculature or skin. Sutures or staples may also be used to hold a membrane-based porcine scaffold in place. The porcine scaffold may be hydrated at the application site during treatment. The porcine scaffold may also be used as an implant. The porcine scaffold can also be used to cover an implant or other device that may be placed on or within a mammalian body.

According to one embodiment, the porcine scaffolds provided herein are useful in conjunction with general surgical procedures to aid in the healing cascade, reduce adhesions and reduce pain/inflammation. Such general surgical procedures include, but are not limited to, breast reconstruction, hernia repair/abdominal wall reconstruction/fascial reconstruction, and vascular bypass graft sites. According to one embodiment, the porcine scaffolds provided herein are useful as hemostasis or biological glues.

According to one embodiment, the porcine scaffolds provided herein are useful for the treatment, reduction and prevention of scar formation. Such scar formation may be the result of trauma or surgical procedure. The surgical procedure includes any procedure that may result in scarring. According to one embodiment, the porcine scaffolds provided herein are useful in neurological surgeries to aid in nerve regeneration or repair, act as a dural substitute, nerve conduit, nerve wrap or in conjunction with aneurysm repair. According to one embodiment, the porcine scaffolds provided herein are useful in orthopedic surgeries (e.g., sports-related injuries to muscle, ligament, and tendons; bone-related surgeries (e.g., spine), total joint replacement, laminectomies (anti-adhesion barrier), tendon/ligament repair, nerve repair, osteoarthritis, cartilage repair, and bone grafting). According to one embodiment, the porcine scaffolds provided herein are useful in colorectal surgery such as colon anastomoses or fistulae repair. According to one embodiment, the porcine scaffolds provided herein are useful in cosmetic surgeries as a dermal filler or to aid in skin wrinkle reduction, skin resurfacing, skin rejuvenation, and other cosmetic purposes.

According to one embodiment, the porcine scaffolds provided herein are useful in cardiovascular surgeries in conjunction with pericardial patch, heart valve leaflets, or vascular graft. According to one embodiment, the porcine scaffolds provided herein are useful in pulmonology for lung repair.

According to one embodiment, the porcine scaffolds provided herein are useful for the treatment and reduction of existing scars (e.g., scar revision). Particularly, the porcine scaffolds provided herein may be used to improve or reduce the appearance of scars, restores skin function and correct skin changes (disfigurement) such as those caused by an injury, wound, or previous surgery. According to one embodiment, the porcine scaffolds described herein can be used as a dressing to aid in the healing and prevention of scars such as those associated with cancer removal (e.g., Moh's surgery).

According to one embodiment, the porcine scaffolds provided herein are useful for the treatment of defects in the ear, nose, mouth or throat such as in the treatment of oral fistulae or septum repair. In some embodiments, the porcine scaffolds provided herein are useful for the treatment of dental defects such as in the wrapping of dental implants, treatment of advanced gingival recession defect, soft palette reconstruction, periodontal defects, or guided tissue repair. According to one embodiment, the porcine scaffolds provided herein are useful for the treatment of ophthalmological conditions (e.g., ocular surface repair, keratitis, corneal ulcer/shield, or pteygium). According to one embodiment, the porcine scaffolds provided herein are useful for the treatment of various gynecological or urological applications such as in ureteral repair, hysterectomy, uterine fibrosis, urinary incontinence, or vaginal prolapse.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### Example 1

### Porcine Cytokine Detection and Quantification Native Porcine Placental Membrane Versus Porcine Scaffold

Cytokine quantification was carried out on the following: (1) porcine scaffolds prepared from porcine placental membrane isolated from three sows from each of three breeds; and (2) native porcine placental membrane isolated from three different sows from the same three different breeds. The porcine scaffold samples were prepared according to the methods provided herein.

The porcine samples were prepared for quantification by creating a lysate of each individual sample. For each sample, the tissue was placed in a microcentrifuge tube with 6 ml of lysis buffer with protease inhibitor from RayBiotech of Norcross, Georgia, USA and allowed to incubate overnight at 4 degrees Celsius. The tissue was homogenized for 2 minutes at 4 degrees Celsius. The microcentrifuge tube was placed into a centrifuge at 10,400 rpm for 20 minutes at 4 degrees Celsius. The resultant supernatant was removed into a micro-Eppendorf tube. The supernatant underwent protein quantification that was carried out using a BCA Protein Quantification Kit from RayBiotech of Norcross, Georgia, USA to assess the total content of protein in the supernatant solution. The samples were diluted to a standard concentration prior to use for cytokine quantification.

Cytokine quantification was carried out utilizing the Quantibody® array from RayBiotech of Norcross, Georgia, USA. The Quantibody® array was specifically utilized to detect standard cytokines thought to be present in porcine placental membrane used to produce porcine scaffolds when processed according to the methods provided herein. The lowest level of detection (referred to as “LOD”) and maximum detection limit (referred to as “MAX”) for each cytokine tested with the Quantibody® array is provided in Table 1.

TABLE 1

| | LOD | MAX |
|---|---|---|
| Cytokine | (pg/ml) | (pg/ml) |
| Eotaxin-1 | 84.1 | 100000.0 |
| EPO | 1.6 | 40000.0 |
| FGF-21 | 1.6 | 1111.1 |
| Galectin-9 | 2.1 | 1111.1 |
| IFNb | 86.0 | 10000.0 |
| IGF-2 | 90.4 | 100000.0 |
| IL-21 | 9188.0 | 100000.0 |
| IL-28B | 513.3 | 100000.0 |
| PIGF-2 | 0.8 | 1000.0 |
| SCF | 1.7 | 3333.3 |
| ANG-1 | 37.4 | 4000.0 |
| IL-17F | 594.2 | 100000.0 |
| MIF | 0.6 | 1333.3 |
| OPG | 11.5 | 6666.7 |
| PDGF-BB | 1.7 | 3333.3 |
| RANTES | 1.0 | 2963.0 |
| TGFa | 0.2 | 222.2 |
| TIMP-1 | 0.2 | 1111.1 |
| TIMP-2 | 1.4 | 10000.0 |
| VEGF | 1.1 | 1000.0 |
| Decorin | 2.3 | 1333.3 |
| GASP-1 | 3.0 | 1333.3 |
| IGFBP-5 | 22.9 | 13333.3 |
| IL-15 | 18.8 | 1481.5 |
| IL-22 | 171.8 | 100000.0 |
| Insulin | 16.6 | 33333.3 |
| IP-10 | 20.4 | 1481.5 |
| MCP-1 | 0.6 | 111.1 |
| NCAM-1 | 123.2 | 100000.0 |
| TWEAK R | 61.9 | 100000.0 |
| CCL3L1 | 15.5 | 2000.0 |
| IFNa | 205.3 | 40000.0 |
| IL-1a | 1125.6 | 40000.0 |
| IL-1ra | 5184.3 | 100000.0 |
| IL-13 | 89.5 | 10000.0 |
| IL-17a | 3.8 | 333.3 |
| IL-18 | 2190.5 | 200000.0 |
| MIG | 8.5 | 1000.0 |
| MIP-1b | 2.4 | 1000.0 |
| PECAM-1 | 6.8 | 13333.3 |
| IL-1b | 7.9 | 3333.3 |
| IL-4 | 2.5 | 1333.3 |
| IL-6 | 0.7 | 666.7 |
| IL-8 | 0.1 | 148.1 |
| IL-10 | 0.9 | 370.4 |
| IL-12p40p70 | 37.9 | 100000.0 |
| GM-CSF | 1.6 | 4444.4 |
| IFNg | 0.6 | 20000.0 |
| TFGb1 | 1006.4 | 100000.0 |
| TNFa | 0.4 | 2222.2 |

The Quantibody® array multiplexed sandwich ELISA-based quantitative array platform was able to simultaneously determine the concentration of multiple cytokines typically present in porcine tissue. The Quantibody® array utilized a pair of cytokine specific antibodies for detection. A capture antibody was first bound to a glass surface. After incubation with the sample porcine tissue, the target cytokine was trapped on the solid surface. A second biotin-labeled detection antibody was then added, which could recognize a different epitope of the target cytokine. The cytokine-antibody-biotin complex was then visualized through the addition of the streptavidin-conjugated Cy3 equivalent dye, using a laser scanner.

The quantification results for each cytokine detected in the porcine scaffolds are provided in Tables 2 and 3 for each of the nine sows (Sow A-Sow I).

TABLE 2

| | Sow A | Sow B | Sow C | Sow D | Sow E |
|---|---|---|---|---|---|
| Cytokine | (pg/ml) | (pg/ml) | (pg/ml) | (pg/ml) | (pg/ml) |
| Eotaxin-1 | 681.9 | 760.5 | 185.2 | 1971.1 | 312.1 |
| EPO | 0.0 | 0.0 | 0.0 | 5.9 | 0.0 |
| FGF-21 | 0.0 | 11.1 | 3.9 | 17.3 | 2.3 |
| Galectin-9 | 0.0 | 3.4 | 0.2 | 2.7 | 4.5 |
| IFNb | 0.0 | 0.0 | 0.0 | 0.0 | 0.0 |
| IGF-2 | 1047.8 | 1902.9 | 1566.2 | 2179.9 | 2103.6 |
| IL-21 | 0.0 | 12497.2 | 2187.0 | 1642.2 | 5681.3 |
| IL-28B | 0.0 | 0.0 | 0.0 | 0.0 | 23.0 |
| PIGF-2 | 17.0 | 126.9 | 43.9 | 22.1 | 51.4 |
| SCF | 0.0 | 15.2 | 3.0 | 14.9 | 8.3 |
| ANG-1 | 38.8 | 8.8 | 0.0 | 0.0 | 0.0 |
| IL-17F | 0.0 | 0.0 | 0.0 | 0.0 | 604.9 |
| MIF | 3.6 | 12.7 | 7.0 | 10.5 | 5.7 |
| OPG | 6.8 | 0.0 | 0.0 | 0.0 | 20.8 |
| PDGF-BB | 1344.6 | 1672.1 | 1447.5 | 2586.4 | 2930.3 |
| RANTES | 0.0 | 0.0 | 0.0 | 0.0 | 0.9 |
| TGFa | 0.0 | 0.0 | 0.0 | 0.0 | 0.3 |
| TIMP-1 | 0.0 | 0.0 | 0.9 | 0.0 | 1.0 |
| TIMP-2 | 21.1 | 30.8 | 12.1 | 19.3 | 59.8 |
| VEGF | 236.6 | 1057.0 | 486.1 | 215.1 | 519.1 |
| Decorin | 147.7 | 689.6 | 1008.2 | 407.5 | 424.3 |
| GASP-1 | 0.0 | 0.0 | 0.0 | 0.0 | 0.0 |
| IGFBP-5 | 0.0 | 0.0 | 2.7 | 12.3 | 6.3 |
| IL-15 | 0.0 | 0.0 | 0.0 | 0.0 | 16.4 |
| IL-22 | 0.0 | 637.1 | 358.4 | 571.0 | 382.8 |
| Insulin | 0.0 | 0.0 | 0.0 | 0.0 | 9.2 |
| IP-10 | 0.0 | 0.0 | 0.0 | 0.0 | 7.0 |
| MCP-1 | 0.0 | 1.0 | 0.9 | 0.8 | 2.5 |
| NCAM-1 | 0.0 | 77.2 | 18.4 | 26.2 | 212.9 |
| TWEAK R | 123.0 | 390.7 | 382.6 | 283.4 | 778.1 |
| CCL3L1 | 19.5 | 19.5 | 4.2 | 35.3 | 28.5 |
| IFNa | 0.0 | 0.0 | 0.0 | 0.0 | 0.0 |
| IL-1a | 0.0 | 0.0 | 0.0 | 94.6 | 0.0 |
| IL-1ra | 0.0 | 0.0 | 0.0 | 0.0 | 0.0 |
| IL-13 | 0.0 | 0.0 | 0.0 | 0.0 | 0.0 |
| IL-17a | 0.0 | 0.0 | 0.0 | 0.0 | 0.0 |
| IL-18 | 212.2 | 1799.7 | 0.0 | 85.5 | 0.0 |
| MIG | 0.0 | 0.4 | 0.7 | 0.0 | 27.7 |
| MIP-1b | 0.0 | 0.0 | 0.0 | 0.0 | 0.0 |
| PECAM-1 | 0.0 | 35.1 | 32.9 | 0.0 | 18.0 |
| IL-1b | 0.0 | 0.8 | 0.0 | 0.0 | 0.0 |
| IL-4 | 0.0 | 0.0 | 0.0 | 2.2 | 0.0 |
| IL-6 | 3.0 | 3.5 | 1.6 | 3.9 | 1.3 |
| IL-8 | 0.1 | 0.1 | 0.0 | 0.0 | 0.0 |
| IL-10 | 0.0 | 0.6 | 0.0 | 0.1 | 0.7 |
| IL-12p40p70 | 168.1 | 89.4 | 24.6 | 0.0 | 242.5 |
| GM-CSF | 0.0 | 3.7 | 0.0 | 5.2 | 10.6 |
| IFNg | 1.7 | 7.8 | 6.5 | 10.9 | 16.9 |
| TGF-β1 | 1280.4 | 4937.4 | 626.2 | 4068.4 | 1732.7 |
| TNFa | 0.0 | 0.0 | 0.0 | 0.0 | 0.0 |

TABLE 3

| | Sow F | Sow G | Sow H | Sow I |
|---|---|---|---|---|
| Cytokine | (pg/ml) | (pg/ml) | (pg/ml) | (pg/ml) |
| Eotaxin-1 | 2455.9 | 1566.6 | 2325.4 | 2356.6 |
| EPO | 4.5 | 0.0 | 0.0 | 10.2 |
| FGF-21 | 11.1 | 0.0 | 9.1 | 5.9 |
| Galectin-9 | 3.8 | 0.0 | 13.1 | 0.0 |
| IFNb | 0.0 | 0.0 | 0.0 | 0.0 |
| IGF-2 | 1887.9 | 1276.8 | 1580.4 | 1554.9 |
| IL-21 | 6160.0 | 0.0 | 36370.5 | 7181.3 |
| IL-28B | 0.0 | 0.0 | 2430.5 | 838.4 |
| PIGF-2 | 82.2 | 64.8 | 50.2 | 91.0 |
| SCF | 12.8 | 0.0 | 28.7 | 13.8 |
| ANG-1 | 86.9 | 0.0 | 112.3 | 0.0 |
| IL-17F | 892.1 | 205.2 | 517.7 | 847.1 |
| MIF | 13.9 | 22.1 | 2.1 | 13.5 |
| OPG | 0.0 | 3.6 | 14.7 | 5.9 |
| PDGF-BB | 1796.9 | 2511.7 | 1828.3 | 2498.5 |
| RANTES | 0.3 | 1.2 | 2.1 | 0.0 |
| TGFa | 0.0 | 0.2 | 0.0 | 0.0 |
| TIMP-1 | 0.0 | 0.2 | 1.0 | 0.6 |
| TIMP-2 | 26.4 | 37.2 | 14.6 | 28.0 |
| VEGF | 497.0 | 241.1 | 146.6 | 353.0 |
| Decorin | 1061.6 | 450.0 | 330.5 | 448.2 |
| GASP-1 | 0.0 | 0.0 | 0.0 | 0.0 |
| IGFBP-5 | 22.6 | 0.0 | 0.0 | 0.0 |
| IL-15 | 3.9 | 0.0 | 0.0 | 0.0 |
| IL-22 | 490.7 | 99.2 | 274.3 | 109.5 |
| Insulin | 0.0 | 0.0 | 0.0 | 0.0 |
| IP-10 | 0.0 | 0.0 | 0.0 | 0.0 |
| MCP-1 | 0.5 | 0.0 | 0.0 | 0.3 |
| NCAM-1 | 103.1 | 0.0 | 0.0 | 0.0 |
| TWEAK R | 434.2 | 24.6 | 114.9 | 120.4 |
| CCL3L1 | 14.9 | 0.0 | 46.7 | 30.8 |
| IFNa | 0.0 | 0.0 | 0.0 | 0.0 |
| IL-1a | 794.4 | 0.0 | 216.8 | 0.0 |
| IL-1ra | 0.0 | 0.0 | 0.0 | 0.0 |
| IL-13 | 0.0 | 0.0 | 0.0 | 0.0 |
| IL-17a | 0.0 | 0.0 | 0.0 | 0.0 |
| IL-18 | 0.0 | 0.0 | 2727.1 | 2702.6 |
| MIG | 4.0 | 0.0 | 4.6 | 0.0 |
| MIP-1b | 1.8 | 0.0 | 0.0 | 0.0 |
| PECAM-1 | 24.9 | 0.0 | 23.6 | 9.2 |
| IL-1b | 0.0 | 0.0 | 0.0 | 8.3 |
| IL-4 | 0.0 | 1.7 | 0.0 | 5.0 |
| IL-6 | 1.7 | 4.4 | 1.3 | 8.6 |
| IL-8 | 0.0 | 0.0 | 0.0 | 0.3 |
| IL-10 | 0.2 | 0.0 | 0.0 | 1.9 |
| IL-12p40p70 | 12.5 | 86.9 | 16.5 | 220.0 |
| GM-CSF | 0.1 | 0.0 | 4.4 | 12.3 |
| IFNg | 2.4 | 9.1 | 7.0 | 11.8 |
| TGF-β1 | 3741.8 | 0.0 | 437.1 | 1557.9 |
| TNFa | 0.5 | 0.0 | 0.5 | 0.0 |

The quantification results for each cytokine detected in the native porcine placental membranes are provided in Tables 4 and 5 for each of the nine sows (Sow J-Sow R).

TABLE 4

| | Sow J | Sow K | Sow L | Sow M | Sow N |
|---|---|---|---|---|---|
| Cytokine | (pg/ml) | (pg/ml) | (pg/ml) | (pg/ml) | (pg/ml) |
| Eotaxin-1 | 0.0 | 0.0 | 0.0 | 0.0 | 0.0 |
| EPO | 0.0 | 0.0 | 0.0 | 15.0 | 0.0 |
| FGF-21 | 0.0 | 0.8 | 0.0 | 0.0 | 0.0 |
| Galectin-9 | 0.0 | 1.0 | 0.0 | 0.0 | 0.0 |
| IFNb | 0.0 | 0.0 | 0.0 | 0.0 | 0.0 |
| IGF-2 | 0.0 | 0.0 | 75.6 | 104.1 | 0.0 |
| IL-21 | 0.0 | 13540.8 | 2897.0 | 0.0 | 0.0 |
| IL-28B | 0.0 | 0.0 | 0.0 | 0.0 | 0.0 |
| PIGF-2 | 421.9 | 335.5 | 422.6 | 330.2 | 365.4 |
| SCF | 7.1 | 0.0 | 7.2 | 0.0 | 0.0 |
| ANG-1 | 184.9 | 86.5 | 112.1 | 50.5 | 112.6 |
| IL-17F | 1,660.2 | 0.0 | 0.0 | 0.0 | 0.0 |
| MIF | 582.2 | 768.5 | 492.9 | 684.1 | 470.6 |
| OPG | 0.0 | 43.5 | 0.0 | 0.0 | 2.1 |
| PDGF-BB | 889.7 | 572.1 | 586.4 | 576.5 | 450.5 |
| RANTES | 0.0 | 0.0 | 0.0 | 0.0 | 0.0 |
| TGFa | 0.5 | 0.8 | 1.0 | 1.1 | 0.8 |
| TIMP-1 | 4.2 | 2.9 | 0.1 | 0.4 | 0.3 |
| TIMP-2 | 1,976.3 | 2,231.2 | 1,880.9 | 1,920.8 | 1,402.1 |
| VEGF | 14.2 | 11.4 | 7.2 | 2.2 | 11.1 |
| Decorin | 471.5 | 491.2 | 502.9 | 445.8 | 453.9 |
| GASP-1 | 97.5 | 57.1 | 139.4 | 96.1 | 42.5 |
| IGFBP-5 | 0.0 | 0.0 | 0.0 | 0.0 | 0.0 |
| IL-15 | 0.0 | 0.0 | 0.0 | 0.0 | 0.0 |
| IL-22 | 0.0 | 145.6 | 0.0 | 240.1 | 90.9 |
| Insulin | 0.0 | 0.0 | 0.0 | 0.0 | 0.0 |
| IP-10 | 0.0 | 0.0 | 0.0 | 0.0 | 0.0 |
| MCP-1 | 70.4 | 50.6 | 52.3 | 44.8 | 57.8 |
| NCAM-1 | 0.0 | 0.0 | 76.0 | 0.0 | 0.0 |
| TWEAK R | 888.6 | 915.2 | 876.5 | 743.3 | 878.8 |
| CCL3L1 | 10.5 | 16.5 | 30.3 | 16.4 | 21.3 |
| IFNa | 507.8 | 280.7 | 329.3 | 285.6 | 155.9 |
| IL-1a | 501.9 | 1,220.6 | 899.3 | 976.3 | 725.8 |
| IL-1ra | 13,522.1 | 23,839.2 | 18,204.8 | 20,715.2 | 19,099.3 |
| IL-13 | 51.1 | 0.0 | 135.7 | 0.0 | 25.9 |
| IL-17a | 0.0 | 2.2 | 2.9 | 0.4 | 2.1 |
| IL-18 | 0.0 | 0.0 | 3.8 | 0.0 | 0.0 |
| MIG | 0.0 | 19.9 | 9.5 | 0.0 | 3.9 |
| MIP-1b | 78.7 | 27.8 | 50.5 | 46.3 | 59.8 |
| PECAM-1 | 4,708.4 | 4,622.0 | 4,677.2 | 4,826.0 | 4,940.6 |
| IL-1b | 0.0 | 0.0 | 0.0 | 0.0 | 0.0 |
| IL-4 | 0.0 | 0.0 | 0.0 | 0.0 | 0.0 |
| IL-6 | 2.6 | 1.5 | 0.9 | 1.5 | 1.2 |
| IL-8 | 1.4 | 0.7 | 0.2 | 0.0 | 0.4 |
| IL-10 | 1.8 | 0.0 | 3.1 | 0.0 | 0.0 |
| IL-12p40p70 | 0.0 | 0.0 | 0.0 | 0.0 | 0.0 |
| GM-CSF | 0.0 | 0.0 | 0.0 | 0.0 | 0.0 |
| IFNg | 0.0 | 0.0 | 0.0 | 0.0 | 0.0 |
| TGF-β1 | 0.0 | 0.0 | 0.0 | 0.0 | 0.0 |
| TNFa | 0.0 | 0.0 | 0.0 | 0.0 | 0.0 |

TABLE 5

| | Sow O | Sow P | Sow Q | Sow R |
|---|---|---|---|---|
| Cytokine | (pg/ml) | (pg/ml) | (pg/ml) | (pg/ml) |
| Eotaxin-1 | 0.0 | 0.0 | 0.0 | 0.0 |
| EPO | 0.0 | 0.0 | 0.0 | 0.0 |
| FGF-21 | 0.0 | 0.0 | 0.0 | 0.0 |
| Galectin-9 | 0.0 | 0.0 | 0.0 | 0.0 |
| IFNb | 0.0 | 0.0 | 0.0 | 0.0 |
| IGF-2 | 0.0 | 0.0 | 0.0 | 0.0 |
| IL-21 | 9,672.2 | 0.0 | 0.0 | 0.0 |
| IL-28B | 0.0 | 0.0 | 0.0 | 0.0 |
| PIGF-2 | 363.6 | 325.6 | 259.0 | 199.4 |
| SCF | 0.0 | 0.0 | 0.0 | 0.0 |
| ANG-1 | 130.3 | 59.0 | 79.6 | 98.7 |
| IL-17F | 256.3 | 2,453.1 | 57.1 | 472.9 |
| MIF | 512.2 | 690.2 | 773.9 | 547.6 |
| OPG | 0.0 | 0.0 | 58.5 | 0.0 |
| PDGF-BB | 411.0 | 408.3 | 388.3 | 416.9 |
| RANTES | 0.0 | 0.0 | 0.0 | 0.0 |
| TGFa | 1.6 | 1.0 | 1.2 | 1.0 |
| TIMP-1 | 0.3 | 2.2 | 1.2 | 2.0 |
| TIMP-2 | 1,749.8 | 2,105.6 | 2,182.9 | 1,639.2 |
| VEGF | 13.3 | 7.9 | 9.4 | 13.4 |
| Decorin | 502.4 | 509.0 | 486.1 | 480.7 |
| GASP-1 | 37.6 | 67.9 | 21.9 | 102.4 |
| IGFBP-5 | 0.0 | 0.0 | 0.0 | 0.0 |
| IL-15 | 0.0 | 0.0 | 0.0 | 0.0 |
| IL-22 | 261.2 | 284.4 | 0.0 | 152.5 |
| Insulin | 0.0 | 0.0 | 0.0 | 0.0 |
| IP-10 | 0.0 | 0.0 | 0.0 | 0.0 |
| MCP-1 | 53.4 | 42.6 | 61.3 | 59.6 |
| NCAM-1 | 142.2 | 48.3 | 0.0 | 0.0 |
| TWEAK R | 890.9 | 754.6 | 855.0 | 872.3 |
| CCL3L1 | 0.0 | 26.8 | 36.1 | 30.5 |
| IFNa | 64.3 | 340.0 | 40.5 | 388.2 |
| IL-1a | 634.8 | 662.3 | 471.8 | 227.5 |
| IL-1ra | 22,370.1 | 17,663.6 | 16,607.3 | 13,500.3 |
| IL-13 | 49.6 | 0.0 | 0.0 | 0.0 |
| IL-17a | 6.2 | 0.0 | 0.0 | 0.0 |
| IL-18 | 0.0 | 0.0 | 0.0 | 0.0 |
| MIG | 18.0 | 2.3 | 0.0 | 0.0 |
| MIP-1b | 43.5 | 26.4 | 49.2 | 64.3 |
| PECAM-1 | 4,868.2 | 4,650.5 | 4,884.0 | 4,677.8 |
| IL-1b | 0.0 | 0.0 | 259.0 | 0.0 |
| IL-4 | 0.0 | 0.0 | 0.0 | 0.0 |
| IL-6 | 1.4 | 0.0 | 0.0 | 0.0 |
| IL-8 | 0.0 | 0.0 | 0.2 | 0.0 |
| IL-10 | 0.0 | 0.0 | 0.0 | 0.0 |
| IL-12p40p70 | 0.0 | 0.0 | 0.0 | 0.0 |
| GM-CSF | 0.0 | 0.0 | 0.0 | 0.0 |
| IFNg | 0.0 | 0.0 | 0.0 | 0.0 |
| TGF-β1 | 0.0 | 0.0 | 0.0 | 0.0 |
| TNFa | 0.0 | 0.0 | 0.0 | 0.0 |

To compare the quantification results of the porcine scaffolds to the native porcine placental membranes, the pg/ml concentrations were converted to mass-to-mass concentrations in pg/mg because: (i) each individual sample had a unique amount of total protein extracted during processing; and (ii) the resultant protein solution was diluted prior to placement into the Quantibody® array. With the known mass of starting tissue for each sow, the protein extraction concentration, and the volume of the dilution, an accurate pg/mg concentration for each cytokine per starting mass of tissue for each sample was calculated. The results of these calculations are provided in Tables 6 through 9.

TABLE 6

| | Sow A | Sow B | Sow C | Sow D | Sow E |
|---|---|---|---|---|---|
| Cytokine | (pg/mg) | (pg/mg) | (pg/mg) | (pg/mg) | (pg/mg) |
| Eotaxin-1 | 150.5 | 152.1 | 39.0 | 437.1 | 77.7 |
| EPO | 0.0 | 0.0 | 0.0 | 1.3 | 0.0 |
| FGF-21 | 0.0 | 2.2 | 0.8 | 3.8 | 0.6 |
| Galectin-9 | 0.0 | 0.7 | 0.0 | 0.6 | 1.1 |
| IFNb | 0.0 | 0.0 | 0.0 | 0.0 | 0.0 |
| IGF-2 | 231.3 | 380.6 | 330.1 | 483.4 | 523.8 |
| IL-21 | 0.0 | 2499.4 | 461.0 | 364.2 | 1414.8 |
| IL-28B | 0.0 | 0.0 | 0.0 | 0.0 | 5.7 |
| PIGF-2 | 3.8 | 25.4 | 9.3 | 4.9 | 12.8 |
| SCF | 0.0 | 3.0 | 0.6 | 3.3 | 2.1 |
| ANG-1 | 8.6 | 1.8 | 0.0 | 0.0 | 0.0 |
| IL-17F | 0.0 | 0.0 | 0.0 | 0.0 | 150.6 |
| MIF | 0.8 | 2.5 | 1.5 | 2.3 | 1.4 |
| OPG | 1.5 | 0.0 | 0.0 | 0.0 | 5.2 |
| PDGF-BB | 296.9 | 334.4 | 305.1 | 573.6 | 729.7 |
| RANTES | 0.0 | 0.0 | 0.0 | 0.0 | 0.2 |
| TGFa | 0.0 | 0.0 | 0.0 | 0.0 | 0.1 |
| TIMP-1 | 0.0 | 0.0 | 0.2 | 0.0 | 0.3 |
| TIMP-2 | 4.7 | 6.2 | 2.6 | 4.3 | 14.9 |
| VEGF | 52.2 | 211.4 | 102.5 | 47.7 | 129.3 |
| Decorin | 32.6 | 137.9 | 212.5 | 90.4 | 105.7 |
| GASP-1 | 0.0 | 0.0 | 0.0 | 0.0 | 0.0 |
| IGFBP-5 | 0.0 | 0.0 | 0.6 | 2.7 | 1.6 |
| IL-15 | 0.0 | 0.0 | 0.0 | 0.0 | 4.1 |
| IL-22 | 0.0 | 127.4 | 75.5 | 126.6 | 95.3 |
| Insulin | 0.0 | 0.0 | 0.0 | 0.0 | 2.3 |
| IP-10 | 0.0 | 0.0 | 0.0 | 0.0 | 1.8 |
| MCP-1 | 0.0 | 0.2 | 0.2 | 0.2 | 0.6 |
| NCAM-1 | 0.0 | 15.4 | 3.9 | 5.8 | 53.0 |
| TWEAK R | 27.2 | 78.1 | 80.6 | 62.9 | 193.8 |
| CCL3L1 | 4.3 | 3.9 | 0.9 | 7.8 | 7.1 |
| IFNa | 0.0 | 0.0 | 0.0 | 0.0 | 0.0 |
| IL-1a | 0.0 | 0.0 | 0.0 | 21.0 | 0.0 |
| IL-1ra | 0.0 | 0.0 | 0.0 | 0.0 | 0.0 |
| IL-13 | 0.0 | 0.0 | 0.0 | 0.0 | 0.0 |
| IL-17a | 0.0 | 0.0 | 0.0 | 0.0 | 0.0 |
| IL-18 | 46.8 | 359.9 | 0.0 | 19.0 | 0.0 |
| MIG | 0.0 | 0.1 | 0.1 | 0.0 | 6.9 |
| MIP-1b | 0.0 | 0.0 | 0.0 | 0.0 | 0.0 |
| PECAM-1 | 0.0 | 7.0 | 6.9 | 0.0 | 4.5 |
| IL-1b | 0.0 | 0.2 | 0.0 | 0.0 | 0.0 |
| IL-4 | 0.0 | 0.0 | 0.0 | 0.5 | 0.0 |
| IL-6 | 0.7 | 0.7 | 0.3 | 0.9 | 0.3 |
| IL-8 | 0.0 | 0.0 | 0.0 | 0.0 | 0.0 |
| IL-10 | 0.0 | 0.1 | 0.0 | 0.0 | 0.2 |
| IL-12p40p70 | 37.1 | 17.9 | 5.2 | 0.0 | 60.4 |
| GM-CSF | 0.0 | 0.7 | 0.0 | 1.2 | 2.7 |
| IFNg | 0.4 | 1.6 | 1.4 | 2.4 | 4.2 |
| TGF-β1 | 282.7 | 987.5 | 132.0 | 902.2 | 431.5 |
| TNFa | 0.0 | 0.0 | 0.0 | 0.0 | 0.0 |

TABLE 7

| | Sow F | Sow G | Sow H | Sow I |
|---|---|---|---|---|
| Cytokine | (pg/mg) | (pg/mg) | (pg/mg) | (pg/mg) |
| Eotaxin-1 | 474.3 | 360.0 | 487.9 | 487.5 |
| EPO | 0.9 | 0.0 | 0.0 | 2.1 |
| FGF-21 | 2.2 | 0.0 | 1.9 | 1.2 |
| Galectin-9 | 0.7 | 0.0 | 2.8 | 0.0 |
| IFNb | 0.0 | 0.0 | 0.0 | 0.0 |
| IGF-2 | 364.6 | 293.4 | 331.6 | 321.7 |
| IL-21 | 1189.7 | 0.0 | 7630.7 | 1485.5 |
| IL-28B | 0.0 | 0.0 | 509.9 | 173.4 |
| PIGF-2 | 15.9 | 14.9 | 10.5 | 18.8 |
| SCF | 2.5 | 0.0 | 6.0 | 2.9 |
| ANG-1 | 16.8 | 0.0 | 23.6 | 0.0 |
| IL-17F | 172.3 | 47.2 | 108.6 | 175.2 |
| MIF | 2.7 | 5.1 | 0.4 | 2.8 |
| OPG | 0.0 | 0.8 | 3.1 | 1.2 |
| PDGF-BB | 347.1 | 577.2 | 383.6 | 516.9 |
| RANTES | 0.1 | 0.3 | 0.4 | 0.0 |
| TGFa | 0.0 | 0.1 | 0.0 | 0.0 |
| TIMP-1 | 0.0 | 0.0 | 0.2 | 0.1 |
| TIMP-2 | 5.1 | 8.6 | 3.1 | 5.8 |
| VEGF | 96.0 | 55.4 | 30.8 | 73.0 |
| Decorin | 205.0 | 103.4 | 69.3 | 92.7 |
| GASP-1 | 0.0 | 0.0 | 0.0 | 0.0 |
| IGFBP-5 | 4.4 | 0.0 | 0.0 | 0.0 |
| IL-15 | 0.8 | 0.0 | 0.0 | 0.0 |
| IL-22 | 94.8 | 22.8 | 57.5 | 22.7 |
| Insulin | 0.0 | 0.0 | 0.0 | 0.0 |
| IP-10 | 0.0 | 0.0 | 0.0 | 0.0 |
| MCP-1 | 0.1 | 0.0 | 0.0 | 0.1 |
| NCAM-1 | 19.9 | 0.0 | 0.0 | 0.0 |
| TWEAK R | 83.9 | 5.7 | 24.1 | 24.9 |
| CCL3L1 | 2.9 | 0.0 | 9.8 | 6.4 |
| IFNa | 0.0 | 0.0 | 0.0 | 0.0 |
| IL-1a | 153.4 | 0.0 | 45.5 | 0.0 |
| IL-1ra | 0.0 | 0.0 | 0.0 | 0.0 |
| IL-13 | 0.0 | 0.0 | 0.0 | 0.0 |
| IL-17a | 0.0 | 0.0 | 0.0 | 0.0 |
| IL-18 | 0.0 | 0.0 | 572.1 | 559.1 |
| MIG | 0.8 | 0.0 | 1.0 | 0.0 |
| MIP-1b | 0.3 | 0.0 | 0.0 | 0.0 |
| PECAM-1 | 4.8 | 0.0 | 5.0 | 1.9 |
| IL-1b | 0.0 | 0.0 | 0.0 | 1.7 |
| IL-4 | 0.0 | 0.4 | 0.0 | 1.0 |
| IL-6 | 0.3 | 1.0 | 0.3 | 1.8 |
| IL-8 | 0.0 | 0.0 | 0.0 | 0.1 |
| IL-10 | 0.0 | 0.0 | 0.0 | 0.4 |
| IL-12p40p70 | 2.4 | 20.0 | 3.5 | 45.5 |
| GM-CSF | 0.0 | 0.0 | 0.9 | 2.6 |
| IFNg | 0.5 | 2.1 | 1.5 | 2.4 |
| TGF-β1 | 722.7 | 0.0 | 91.7 | 322.3 |
| TNFa | 0.1 | 0.0 | 0.1 | 0.0 |

TABLE 8

| | Sow J | Sow K | Sow L | Sow M | Sow N |
|---|---|---|---|---|---|
| Cytokine | (pg/mg) | (pg/mg) | (pg/mg) | (pg/mg) | (pg/mg) |
| Eotaxin-1 | 0.0 | 0.0 | 0.0 | 0.0 | 0.0 |
| EPO | 0.0 | 0.0 | 0.0 | 2.0 | 0.0 |
| FGF-21 | 0.0 | 0.1 | 0.0 | 0.0 | 0.0 |
| Galectin-9 | 0.0 | 0.1 | 0.0 | 0.0 | 0.0 |
| IFNb | 0.0 | 0.0 | 0.0 | 0.0 | 0.0 |
| IGF-2 | 0.0 | 0.0 | 6.2 | 13.5 | 0.0 |
| IL-21 | 0.0 | 1542.3 | 236.1 | 0.0 | 0.0 |
| IL-28B | 0.0 | 0.0 | 0.0 | 0.0 | 0.0 |
| PIGF-2 | 40.6 | 38.2 | 34.4 | 42.9 | 46.9 |
| SCF | 0.7 | 0.0 | 0.6 | 0.0 | 0.0 |
| ANG-1 | 17.8 | 9.9 | 9.1 | 6.6 | 14.4 |
| IL-17F | 191.7 | 0.0 | 0.0 | 0.0 | 0.0 |
| MIF | 56.1 | 89.6 | 40.2 | 89.0 | 60.4 |
| OPG | 0.0 | 5.0 | 0.0 | 0.0 | 0.3 |
| PDGF-BB | 85.7 | 65.2 | 47.8 | 75.0 | 57.8 |
| RANTES | 0.0 | 0.0 | 0.0 | 0.0 | 0.0 |
| TGFa | 0.0 | 0.1 | 0.1 | 0.1 | 0.1 |
| TIMP-1 | 0.4 | 0.3 | 0.0 | 0.1 | 0.0 |
| TIMP-2 | 190.3 | 254.1 | 153.3 | 249.8 | 179.8 |
| VEGF | 1.4 | 1.3 | 0.6 | 0.3 | 1.4 |
| Decorin | 45.4 | 55.9 | 41.0 | 58.0 | 58.2 |
| GASP-1 | 9.4 | 6.5 | 11.4 | 12.5 | 5.5 |
| IGFBP-5 | 0.0 | 0.0 | 0.0 | 0.0 | 0.0 |
| IL-15 | 0.0 | 0.0 | 0.0 | 0.0 | 0.0 |
| IL-22 | 0.0 | 16.6 | 0.0 | 31.2 | 11.7 |
| Insulin | 0.0 | 0.0 | 0.0 | 0.0 | 0.0 |
| IP-10 | 0.0 | 0.0 | 0.0 | 0.0 | 0.0 |
| MCP-1 | 6.8 | 5.8 | 4.3 | 5.8 | 7.4 |
| NCAM-1 | 0.0 | 0.0 | 6.2 | 0.0 | 0.0 |
| TWEAK R | 85.6 | 104.2 | 71.4 | 96.7 | 112.7 |
| CCL3L1 | 1.0 | 1.9 | 2.5 | 2.1 | 2.7 |
| IFNa | 48.9 | 32.0 | 26.8 | 37.1 | 20.0 |
| IL-1a | 48.3 | 139.0 | 73.3 | 127.0 | 93.1 |
| IL-1ra | 1302.2 | 2715.3 | 1483.7 | 2694.0 | 2449.5 |
| IL-13 | 4.9 | 0.0 | 11.1 | 0.0 | 3.3 |
| IL-17a | 0.0 | 0.3 | 0.2 | 0.1 | 0.3 |
| IL-18 | 0.0 | 0.0 | 0.3 | 0.0 | 0.0 |
| MIG | 0.0 | 2.3 | 0.8 | 0.0 | 0.5 |
| MIP-1b | 7.6 | 3.2 | 4.1 | 6.0 | 7.7 |
| PECAM-1 | 453.4 | 526.4 | 381.2 | 627.6 | 633.6 |
| IL-1b | 0.0 | 0.0 | 0.0 | 0.0 | 0.0 |
| IL-4 | 0.0 | 0.0 | 0.0 | 0.0 | 0.0 |
| IL-6 | 0.3 | 0.2 | 0.1 | 0.2 | 0.2 |
| IL-8 | 0.1 | 0.1 | 0.0 | 0.0 | 0.0 |
| IL-10 | 0.2 | 0.0 | 0.3 | 0.0 | 0.0 |
| IL-12p40p70 | 0.0 | 0.0 | 0.0 | 0.0 | 0.0 |
| GM-CSF | 0.0 | 0.0 | 0.0 | 0.0 | 0.0 |
| IFNg | 0.0 | 0.0 | 0.0 | 0.0 | 0.0 |
| TGF-β1 | 0.0 | 0.0 | 0.0 | 0.0 | 0.0 |
| TNFa | 0.0 | 0.0 | 0.0 | 0.0 | 0.0 |

TABLE 9

| | Sow O | Sow P | Sow Q | Sow R |
|---|---|---|---|---|
| Cytokine | (pg/mg) | (pg/mg) | (pg/mg) | (pg/mg) |
| Eotaxin-1 | 0.0 | 0.0 | 0.0 | 0.0 |
| EPO | 0.0 | 0.0 | 0.0 | 0.0 |
| FGF-21 | 0.0 | 0.0 | 0.0 | 0.0 |
| Galectin-9 | 0.0 | 0.0 | 0.0 | 0.0 |
| IFNb | 0.0 | 0.0 | 0.0 | 0.0 |
| IGF-2 | 0.0 | 0.0 | 0.0 | 0.0 |
| IL-21 | 1273.8 | 0.0 | 0.0 | 0.0 |
| IL-28B | 0.0 | 0.0 | 0.0 | 0.0 |
| PIGF-2 | 47.9 | 37.4 | 36.7 | 24.2 |
| SCF | 0.0 | 0.0 | 0.0 | 0.0 |
| ANG-1 | 17.2 | 6.8 | 11.3 | 12.0 |
| IL-17F | 33.8 | 282.0 | 8.1 | 57.5 |
| MIF | 67.5 | 79.3 | 109.7 | 66.6 |
| OPG | 0.0 | 0.0 | 8.3 | 0.0 |
| PDGF-BB | 54.1 | 46.9 | 55.0 | 50.7 |
| RANTES | 0.0 | 0.0 | 0.0 | 0.0 |
| TGFa | 0.2 | 0.1 | 0.2 | 0.1 |
| TIMP-1 | 0.0 | 0.3 | 0.2 | 0.2 |
| TIMP-2 | 230.4 | 242.0 | 309.3 | 199.2 |
| VEGF | 1.8 | 0.9 | 1.3 | 1.6 |
| Decorin | 66.2 | 58.5 | 68.9 | 58.4 |
| GASP-1 | 5.0 | 7.8 | 3.1 | 12.4 |
| IGFBP-5 | 0.0 | 0.0 | 0.0 | 0.0 |
| IL-15 | 0.0 | 0.0 | 0.0 | 0.0 |
| IL-22 | 34.4 | 32.7 | 0.0 | 18.5 |
| Insulin | 0.0 | 0.0 | 0.0 | 0.0 |
| IP-10 | 0.0 | 0.0 | 0.0 | 0.0 |
| MCP-1 | 7.0 | 4.9 | 8.7 | 7.2 |
| NCAM-1 | 18.7 | 5.6 | 0.0 | 0.0 |
| TWEAK R | 117.3 | 86.7 | 121.2 | 106.0 |
| CCL3L1 | 0.0 | 3.1 | 5.1 | 3.7 |
| IFNa | 8.5 | 39.1 | 5.7 | 47.2 |
| IL-1a | 83.6 | 76.1 | 66.9 | 27.7 |
| IL-1ra | 2946.1 | 2030.4 | 2353.3 | 1641.0 |
| IL-13 | 6.5 | 0.0 | 0.0 | 0.0 |
| IL-17a | 0.8 | 0.0 | 0.0 | 0.0 |
| IL-18 | 0.0 | 0.0 | 0.0 | 0.0 |
| MIG | 2.4 | 0.3 | 0.0 | 0.0 |
| MIP-1b | 5.7 | 3.0 | 7.0 | 7.8 |
| PECAM-1 | 641.1 | 534.6 | 692.1 | 568.6 |
| IL-1b | 0.0 | 0.0 | 36.7 | 0.0 |
| IL-4 | 0.0 | 0.0 | 0.0 | 0.0 |
| IL-6 | 0.2 | 0.0 | 0.0 | 0.0 |
| IL-8 | 0.0 | 0.0 | 0.0 | 0.0 |
| IL-10 | 0.0 | 0.0 | 0.0 | 0.0 |
| IL-12p40p70 | 0.0 | 0.0 | 0.0 | 0.0 |
| GM-CSF | 0.0 | 0.0 | 0.0 | 0.0 |
| IFNg | 0.0 | 0.0 | 0.0 | 0.0 |
| TGF-β1 | 0.0 | 0.0 | 0.0 | 0.0 |
| TNFa | 0.0 | 0.0 | 0.0 | 0.0 |

The mass-to-mass values were used to statistically compare the concentrations of the cytokines of the porcine scaffolds to the native porcine placental membranes. Six cytokines that were detectable in all 18 samples had a majority of samples in the best confidence range of the Quantibody® array and demonstrated statically significant differences (p<0.05) (see Table 10).

TABLE 10

| | | | Native Porcine | |
|---|---|---|---|---|
| | Porcine | | Placental | |
| | Scaffold | | Membrane | |
| | Mean ± SD | % in Best | Mean ± SD | % in Best |
| Cytokine | (pg/mg) | Confidence | (pg/mg) | Confidence |
| Decorin | 116.6 ± 59.5 | 100% | 56.7 ± 8.8 | 100% |
| MIF | 2.2 ± 1.4 | 100% | 73.1 ± 20.9 | 100% |
| PDGF-BB | 451.6 ± 152.9 | 100% | 59.8 ± 13.1 | 100% |
| TIMP-2 | 6.1 ± 3.7 | 100% | 223.2 ± 47.3 | 100% |
| VEGF | 88.7 ± 55.5 | 88.9% | 1.2 ± 0.5 | 100% |
| PIGF-2 | 12.9 ± 6.8 | 88.9% | 38.8 ± 7.1 | 88.9% |

The process used to create the porcine scaffolds from the native porcine placental membrane greatly increases the concentration of decorin, platelet derived growth factor-BB (PDGF-BB), and vascular endothelial growth factor (VEGF). Conversely, the process used to create the porcine constructs from the native porcine placental membrane greatly decreases the concentration of macrophage migration inhibitory factor (MIF), tissue inhibitor of metalloproteinase 2 (TIMP-2), and placental growth factor 2 (PIGF-2). These six factors are all known to have an impact on cutaneous wound healing.

Decorin, a proteoglycan, binds to Collagen Type I and has a role in extracellular matrix assembly. A study investigating the role of decorin in cutaneous wound healing found increased vessel formation and fibrovascular ingrowth of implanted sponges (Jarvelainen 2006). The increased concentration of decorin in the porcine scaffolds provides a promotive effect on wound healing following placement by increasing vessel formation at a wound site.

PDGF-BB is a growth factor known to regulate cell growth and division. A recombinant version of the protein has successfully treated chronic wounds (Wieman 1998). A study examining the activity and concentration of cytokines in non-healing and healing chronic leg ulcers found that a high concentration of PDGF was seen in healing wounds when compared to non-healing wounds (Trengrove, 2001). Multiple studies have shown that PDGF has an influence on platelets, keratinocytes, macrophages, endothelial cells, and fibroblasts (Barrientos 2008), while specifically influencing matrix formation and remodeling (Heldin 1999, Lederle 2006, Uutela 2004). The increased concentration of PDGF-BB in the porcine scaffolds may influence multiple cell types that play a role in the healing response.

VEGF is a growth factor known to stimulate new blood vessel formation. A study examining the activity and concentration of cytokines in non-healing and healing chronic leg ulcers found that high concentration of VEGF was seen in healing wounds when compared to non-healing wounds (Trengrove 2001). Studies have shown that VEGF has an influence on platelets, neutrophils, macrophages, endothelial cells, and fibroblasts (Barrientos 2008), while specifically influencing angiogenesis (Johnson 2014, Jazwa 2006, Thomas 1996) and granulation tissue formation (Yerba 1996, Nissen 1998). The increased concentration of VEGF in the porcine scaffolds may influence multiple cell types to an early regenerative response.

MIF, a proinflammatory cytokine and regulator of innate immunity, has a complex role in cutaneous wound healing. Studies have shown that MIF can elicit both reparative and inflammatory responses (Gilliver 2010). Studies have demonstrated that MIF plays an important role in early inflammatory response but higher levels later in wound healing can inhibit the process (Ashcroft 2003, Shimizu 2005). The decreased concentration of MIF in the porcine scaffolds may have a positive effect on inflammatory modulation during wound healing.

TIMP-2, an inhibitor of matrix metalloproteinase (MMP) activity, plays an important role in extracellular matrix homeostasis by preventing MMPs from breakdown the ECM proteins. TIMP-2, unlike other TIMPs, has been shown to inhibit the proliferation of endothelial cells (Murphy 1993). The decreased concentration of TIMP-2 in the porcine scaffold may allow the inhibitor action on MMPs without the negative effect on endothelial cell proliferation.

PIGF-2, a growth factor that is member of the VEGF sub-family, plays a key role in angiogenesis and vasculogenesis, particularly during embryogenesis. Studies have shown that PIGF is produced by keritinocytes during wound healing (Failla 2000) and stimulates collagen production by fibroblasts (Arif 2020). The decreased concentration, but not complete removal of PIGF-2, in the porcine scaffold may stimulate collagen production during wound healing.

It is also noted that the process used to create the porcine scaffolds statistically significantly increases the concentration of transforming growth factor-β1 (TGF-β1) and insulin like growth factor-2 (IGF-2), such that they are detectable in the assays of the porcine scaffolds but below the level of detection or best confidence range of the assays of the native porcine placental membrane. These two growth factors are known to have an impact on cutaneous wound healing. Comparisons of the two cytokines are provided in Table 11.

TABLE 11

| | | | Native Porcine | |
|---|---|---|---|---|
| | Porcine | | Placental | |
| | Scaffold | | Membrane | |
| | Mean ± SD | % in Best | Mean ± SD | % in Best |
| Cytokine | (pg/mg) | Confidence | (pg/mg) | Confidence |
| TGF-β1 | 430.3 ± 360.7 | 33.3% | 0 ± 0 | 0% |
| IGF-2 | 362.3 ± 91.3 | 100% | 2.2 ± 4.7 | 0% |

The TGF-β superfamily of growth factors, including TGF-β1, is known to regulate multiple cellular functions including cell growth, cell proliferation, and cell differentiation. A study examined TGF-β1's impact on various aspects of wound healing and demonstrated that increased in vitro proliferation of fibroblasts occurred with application of the growth factor (Rolfe 2007). A second study highlighted the role that TGF-β plays in stimulating production of granulation tissue (Singer 1999). Multiple studies have shown that TGF-β has an influence on platelets, keratinocytes, macrophages, lymphocytes, and fibroblasts (Barrientos 2008), while specifically influencing extracellular matrix formation and remodeling (Tsunawaki 1988, Riedel 2007). The increased concentration of TGF-β1 in the porcine scaffold could influence multiple cell types to a healing response.

IGF-2 is a growth factor known to regulate cell growth, especially during fetal development. A study demonstrated that IGF-2 expression increases from initial injury until 10 to 15 days post injury in a rat model of wound healing (Gartner 1992). A second study confirmed that IGF-2 has increasing expression from initial injury and is expressed at higher levels in diabetic animals. The study also localized the IGF-2 to the epithelium of healing wounds (Brown 1997). The increased concentration of IGF-2 in the porcine scaffold could influence epithelial healing.

We claim:

1. A porcine scaffold comprising:
decellularized, porcine placental extracellular matrix,
wherein the porcine scaffold comprises at least one cytokine present in an amount different from native porcine placental membrane.

2. The porcine scaffold of claim 1, wherein the at least one cytokine is decorin present in an amount of at least about 70 pg/mg.

3. The porcine scaffold of claim 1, wherein the at least one cytokine is MIF present in an amount of less than about 50 pg/mg.

4. The porcine scaffold of claim 1, wherein the at least one cytokine is PDGF-BB present in an amount of at least about 75 pg/mg.

5. The porcine scaffold of claim 1, wherein the at least one cytokine is TIMP-2 present in an amount of less than about 175 pg/mg.

6. The porcine scaffold of claim 1, wherein the at least one cytokine is VEGF present in an amount of at least about 3 pg/mg.

7. The porcine scaffold of claim 1, wherein the at least one cytokine is PIGF-2 present in an amount of less than about 30 pg/mg.

8. The porcine scaffold of claim 1, wherein the at least one cytokine is TGF-β1 in an amount of at least about 82 pg/mg.

9. The porcine scaffold of claim 1, wherein the at least one cytokine is IGF-2 in an amount of at least about 7 pg/mg.

10. The porcine scaffold of claim 1, wherein the dehydrated porcine placental membrane is treated with a bioburden reduction step, a detergent rinse step, and a viral inactivation step.

11. A porcine scaffold comprising:
decellularized, porcine placental extracellular matrix,
wherein the porcine scaffold comprises at least one cytokine for increasing vessel formation at a wound site.

12. The porcine scaffold of claim 11, wherein the at least one cytokine is decorin present in an amount of at least about 70 pg/mg.

13. The porcine scaffold of claim 11, wherein the at least one cytokine is VEGF present in an amount of at least about 3 pg/mg.

14. The porcine scaffold of claim 11, wherein the at least one cytokine is PIGF-2 present in an amount of less than about 30 pg/mg.

15. A porcine scaffold comprising:
decellularized, porcine placental extracellular matrix,
wherein the porcine scaffold comprises at least one cytokine to regulate cell growth and division.

16. The porcine scaffold of claim 15, wherein the at least one cytokine is PDGF-BB present in an amount of at least about 75 pg/mg.

17. The porcine scaffold of claim 15, wherein the at least one cytokine is TGF-β1 in an amount of at least about 82 pg/mg.

18. The porcine scaffold of claim 15, wherein the at least one cytokine is IGF-2 in an amount of at least about 7 pg/mg.

19. A porcine scaffold comprising:
decellularized, porcine placental extracellular matrix,
wherein the porcine scaffold comprises at least one cytokine that inhibits matrix metalloproteinase activity.

20. The porcine scaffold of claim 19, wherein the cytokine is TIMP-2 present in an amount of less than about 175 pg/mg.

21. A porcine scaffold comprising:
decellularized, porcine placental extracellular matrix,
wherein the porcine scaffold comprises at least one cytokine for inflammatory modulation.

22. The porcine scaffold of claim 21, wherein the cytokine is MIF present in an amount of less than about 50 pg/mg.

23. A porcine scaffold comprising:
decellularized, porcine placental extracellular matrix,
wherein the porcine scaffold comprises at least one cytokine for stimulating collagen production.

24. The porcine scaffold of claim 23, wherein the cytokine is PIGF-2 present in an amount of less than about 30 pg/mg.

25. A method of treating a defect, the method comprising the step of:
administering a porcine scaffold as provided in claim 1 to a defect.

26. The method of claim 25, wherein the defect is selected from a partial thickness wound, full thickness wound, pressure ulcer, venous ulcer, diabetic ulcer, chronic vascular ulcer, tunneled or undermined wound, surgical wound, wound dehiscence, abrasion, laceration, second degree burn, skin tear, and draining wound.

27. A wound dressing comprising a porcine scaffold as in claim 1.

28. The wound dressing of claim 27, wherein the porcine scaffold includes a surface defining one or more fenestrations.
